(19) United States
(12) Patent Application Publication
(10) Pub. No.: US 2006/0045818 A1
(43) Pub. Date: Mar. 2, 2006

(54) Combination coil type lamp and fragrance producing cartridge

(76) Inventor: Gregory Moreland, Mission Hills, CA (US)

Correspondence Address: GENE SCOTT; PATENT LAW & VENTURE GROUP, 3140 RED HILL AVENUE, SUITE 150, COSTA MESA, CA, 92626-3440, US

(21) Appl. No.: 10/933,743

(22) Filed: Sep. 2, 2004

(51) Int. Cl. A61L 9/03

(52) U.S. Cl. 422/125

(57) ABSTRACT

A fragrance producing apparatus comprises, in combination, an illuminating device having, at a proximal end, a base adapted for engaging a lamp socket, and a lamp extending from the base. The lamp provides a cylindrical coil portion and a linear portion. The linear portion is positioned diametrically across the distal end of the coil portion. A fragrance producing packet contains a pouch holding a supply of a fragrance agent. The fragrance producing packet is engaged with the lamp wherein the fragrance agent is heated by the lamp when the illuminating device is illuminated, the fragrance producing packet is adapted for reacting to heat by slowly emitting the fragrance agent. A tab on the packet grips the linear portion of the lamp and places the packet suspended within the coil portion of the lamp.

### BACKGROUND OF THE INVENTION

1. Related Applications

none

2. Incorporation by Reference

Applicant hereby incorporates herein by reference, U.S. Pat. Nos. 6,254,248, 6,035,098, 5,908,231 and 4,965,490.

3. Field of the Invention

This invention relates generally to fragrance producing apparatus and especially of the type that are heated so as to enable a fragrance agent to liberated in a controlled manner.

4. Description of Related Art

The following art defines the present state of this field and each disclosure is hereby incorporated herein by reference:

McAuley et al., U.S. Pat. No. 6,254,248, describes a fragrance dispenser for use with a light bulb and fragrance medium that emits a fragrance when heated. The fragrance dispenser includes a body member having an opening for receiving a portion of a light bulb and having a cavity for holding a fragrance medium so that when the fragrance medium is held in the cavity, the light bulb is energized, and at least a portion of the light bulb is received in the opening the body member, heat from the light bulb will be transferred through at least a portion of the body member to the fragrance medium held in the cavity in the body member to cause the fragrance medium to emit a fragrance. The fragrance dispenser additionally includes control structure for controlling the emissions of fragrance from the fragrance medium held in the cavity in the body member.

Chipalkatti et al., U.S. Pat. No. 6,035,098, describes a scent-bearing ring adaptable for at least semi-permanent engagement with the neck of an incandescent bulb that generates both light and heat. The ring comprises an annular body having an inner layer formed to engage the bulb neck and an outer layer containing a scent or vapor-releasing agent actuated by heat. The inner layer is formed from a moldable material at least capable of resisting without substantial damage the heat generated by the bulb when the bulb is operated in a base-down position and capable of transferring the heat to the outer layer, the outer layer being moldable and porous.

Huff, U.S. Pat. No. 5,908,231, describes an improved fragrance dispenser of safe, simple and inexpensive design that is reusable and can be utilized on a light bulb regardless of the orientation of the bulb. The fragrance dispenser securely grips the light bulb at its widest point such that the light bulb may be oriented in any direction. Thus the fragrance dispenser may be used, for example, on a telescoping desk lamp or a swinging lamp where the bulb can be oriented in any direction not merely base up or base down. The fragrance dispenser is made from absorbent material that is non-combustible and reusable, such that the absorbed fragrance oil is vaporized to scent the room air with a pleasing fragrance when the light bulb is turned on and heat is generated.

Ratner, U.S. Pat. No. 4,965,490, describes a vapor-generating electric lamp which includes a sealed lamp envelope having a concave depression therein, an incandescent filament mounted within the lamp envelope, a lamp base, and a replaceable, solid insert in the concave depression. The insert produces a scent when it is warmed by heat from the filament. Preferably, the insert is a polyamide impregnated with a scent and includes a portion that matches the shape of the depression in the lamp envelope. The depression for holding the insert is preferably located at the opposite end of the lamp from the base. When the lamp is used in a base-down orientation, the insert is held in the depression by gravity.

Spector, U.S. Pat. No. 4,647,433 describes a long-life aroma-generating miniature capsule attachable to the surface of an incandescent light bulb to be activated by heat emanating therefrom. The capsule is constituted by a flexible pad of porous material impregnated with a volatile liquid fragrance, the pad being sandwiched between base and cover plies formed of a metal foil-plastic laminate peripherally joined together to create an envelope whose cavity is occupied by the pad. The cover ply has a vent hole therein, and the outer surface of the base ply is coated with a layer of pressure-sensitive adhesive, whereby the capsule may be conformed and adhered to the contoured surface of the light bulb. The adhered capsule is in heat transfer relation to the bulb, as a consequence of which the liquid impregnant is volatilized to produce an aromatic vapor that is discharged through the vent hole. The metal foil in the envelope acts as a heat radiator to dissipate the heat transferred thereto to a degree reducing the rate of volatilization to a level at which the capsule remains effective as an aroma generator for a prolonged period before the liquid is exhausted.

Gyulay, U.S. Pat. No. 4,647,428 describes a room air freshener in the form of a porous ceramic ring in combination with an uprightly positioned light bulb. The ring includes a liquid premeasuring cavity so that a fragrance-generating liquid filling the cavity will be completely absorbed by the ceramic ring. When the liquid filling the cavity is entirely absorbed by the ring, the ring may be placed on a light bulb for vaporization of the absorbed liquid and without risk of spillage regardless of the attitude or levelness of the ring on the bulb. When the lamp bulb is turned on, the ring is heated to vaporize the liquid and release the fragrance which permeates the room.

Spector, U.S. Pat. No. 4,493,011 describes an aroma disc usable in conjunction with a conventional table lamp whose light bulb socket has a harp secured thereto to support a lampshade, the harp being provided with an upwardly projecting bolt on which is received the center collar of the lampshade spider. The aroma disc includes a round pad of porous material impregnated with a liquid fragrance, the pad being sandwiched between two round plies of reflective metal which are peripherally joined together and are provided with a center hole to accommodate the harp bolt whereby when the disc is installed on the lamp it is placed above the bulb. Concentric with the center hole on each ply of the disc is a circular array of openings which define ports to expose the pad. When the lamp is turned on, heated air currents arising from the bulb penetrate the ports and pass through the pad to volatilize the liquid fragrance, thereby generating an aromatic vapor which suffuses the room illuminated by the lamp.

Lindauer, U.S. Pat. No. 4,184,099 describes bodies which are formed compositions of Versalon.RTM. type polyamide resin containing from about 35% up to about 70% by weight of highly volatile materials such as perfumes, odorants, insecticides, bactericides, and animal repellents which are able to act in a very diluted vapor state in the air for relatively long periods of time, are described. The highly volatile substances are released in a controlled manner over a long period of time. The molecular weight of the resin is between 9,000 and 12,000, and its softening point varies from 120.degree. C. up to 400.degree. C. In addition, articles are described incorporating said composition and, as part of the instant invention, a light bulb coated with a toroidal article which consists essentially of said composition, is included. The method of our invention comprises heating Versalon.RTM. polyamide resin (Note 1) having a molecular weight of from 9,000 up to 12,000 until it is pourable and stirrable, e.g., to a temperature of between 120.degree. C. and 400.degree. C., and then blending from 35 up to 70% of a highly volatile ingredient such as a perfume oil, an insecticide composition, a bactericide composition, or an animal repellent composition, into the melt until a uniform mixture is obtained, and cooling the mixture to solidify it as such or in a toroidal state, preferably rapidly as by quenching in cold water. The product so obtained is then formed into an article such as a coating on a substrate such as an electric light bulb. Alternatively, one or more volatile ingredients are blended into the resin in the feed line of a molding machine in which the resin bodies containing said volatile ingredients are fashioned directly.

Moody, Des U.S. 363537 shows the ornamental design for a scented decorative ring for lamps, as shown and described.

Our prior art search with abstracts described above teaches: a controlled fragrance dispenser for a light bulb; a scent lamp; a light bulb fragrance dispenser and a scent generating lamp. Thus, the prior art shows that it is known to provide a fragrance dispenser that emits a desired aroma when heated. It is further known to provide a fragrance pouch that enables a fragrance agent to permeate through a thin wall when heated. Still further, it is known to heat fragrance emitters with a light bulb. However, the prior art fails to teach a pouch that is adapted to be supported within the coil of a lamp and, that is adapted for gripping such a lamp so as to hold the fragrance pouch within the coil. The present invention fulfills these needs and provides further related advantages as described in the following summary.

### SUMMARY OF THE INVENTION

The present invention teaches certain benefits in construction and use which give rise to the objectives described below.

In one of the best mode preferred embodiments of the present invention, a fragrance producing apparatus includes, an illuminating device having, at a proximal end, a base adapted for engaging a lamp socket, and a lamp extending from the base. The lamp provides a cylindrical coil portion and a linear portion. The linear portion is positioned diametrically across the distal end of the coil portion. A fragrance producing packet contains a pouch holding a supply of a fragrance agent. The fragrance producing packet is held within the lamp coil wherein the fragrance agent is heated by the lamp when the illuminating device is illuminated. The fragrance producing packet is adapted for reacting to heat by slowly emitting the fragrance agent. A tab on the packet rests on the coil or grips the linear portion of the lamp and places the packet suspended within the coil portion of the lamp. Of course, alternative configurations for coiled lamps may be used and the packet may be adapted to engage such lamp coils in other ways.

A primary objective of the present invention is to provide an apparatus and method of use of such apparatus that yields advantages not taught by the prior art.

Another objective of the invention is to provide a fragrance producing lamp.

A further objective of the invention is provide such a lamp that is able to emit a fragrance aroma when the space illuminated by the lamp is occupied.

A still further objective of the invention is to provide such a lamp that is able to emit the aroma over an extended period of time, and when exhausted may be replenished.

A still further objective of the invention is to provide such a lamp that is able to accept a fragrance emitter within so as to not diminish light output.

Other features and advantages of the embodiments of the present invention will become apparent from the following more detailed description, taken in conjunction with the accompanying drawings, which illustrate, by way of example, the principles of at least one of the possible embodiments of the invention.

### BRIEF DESCRIPTION OF THE DRAWINGS

The accompanying drawings illustrate at least one of the best mode embodiments of the present invention. In such drawings:

FIG. 1 is a perspective view of one embodiment of a fragrance producing packet of the present invention;

FIG. 2 is a perspective view of a further embodiment of the packet of the present invention;

FIG. 3 is a perspective view of the one embodiment packet as engaged with a coil type lamp of the present invention; and

FIG. 4 is a perspective view of the further embodiment of the packet as engaged with the coil type lamp.

### DETAILED DESCRIPTION OF THE INVENTION

The above described drawing figures illustrate the present invention in at least one of its preferred, best mode embodiments, which is further defined in detail in the following description. Those having ordinary skill in the art may be able to make alterations and modifications in the present invention without departing from its spirit and scope. Therefore, it must be understood that the illustrated embodiments have been set forth only for the purposes of example and that they should not be taken as limiting the invention as defined in the following.

The present invention is a fragrance producing apparatus adapted for engaging a lamp socket (not shown) of any common type, as for instance the threaded type used for common household light bulbs. The apparatus includes an illuminating device 20 and a scenting device 50 joined as a combination that yields the benefits described above. The illuminating device 20 has at a proximal end, a base 30, preferably made of a heat resisting material such as a polyurethane plastic. This base 30 is adapted with a threaded portion 32 for engaging the lamp socket. A lamp 40 is supported by and extends from the base 30. The lamp 40 provides a cylindrical coil portion 42 and a linear portion 44 as is clearly shown in FIG. 4. The linear portion 44 is positioned diametrically across the coil portion 42 at its distal end. Such lamps 40 are manufactured by Phillips Lighting, Osram-Sylvania and the General Electric Company and other world-wide manufacturers/distributors. Further information about this form of lighting is made available by the U.S. EPA, Climate Protection Partnerships Division, ENERGY STAR Programs, 1200 Pennsylvania Ave NW, Washington, D.C. 20460. Such coil lamps are being used widely at this time and provide the advantages of producing a white light similar to natural light, are energy efficient and last almost indefinitely.

The scenting device 50 preferably is a fragrance producing packet which provides a pouch 52 holding a supply of a fragrance agent 54. The fragrance producing packet 50 is preferably made of a somewhat stiff sheet plastic material. However pouch 52 is preferably flexible and comprises a permeable thin film. Packet 50 is engaged with the lamp 40 in such a manner that the fragrance agent 54 is heated by the lamp 40 when it is lit. The fragrance producing packet 50 is adapted for reacting to heat produced by the lamp 40 by slowly emitting the fragrance agent 54 preferably by diffusion through the permeable thin film. Such products are manufactured by S.C. Johnson & Sons of Racine, Wis., and sold under the trademarks: Glade® and Plugins®. Other means of emitting the fragrance agent 54 are well known in the art and may be alternatively used.

Preferably, the pouch 52 of the fragrance producing packet 50 provides a thin wall 56 that is permeable to the fragrance agent 54.

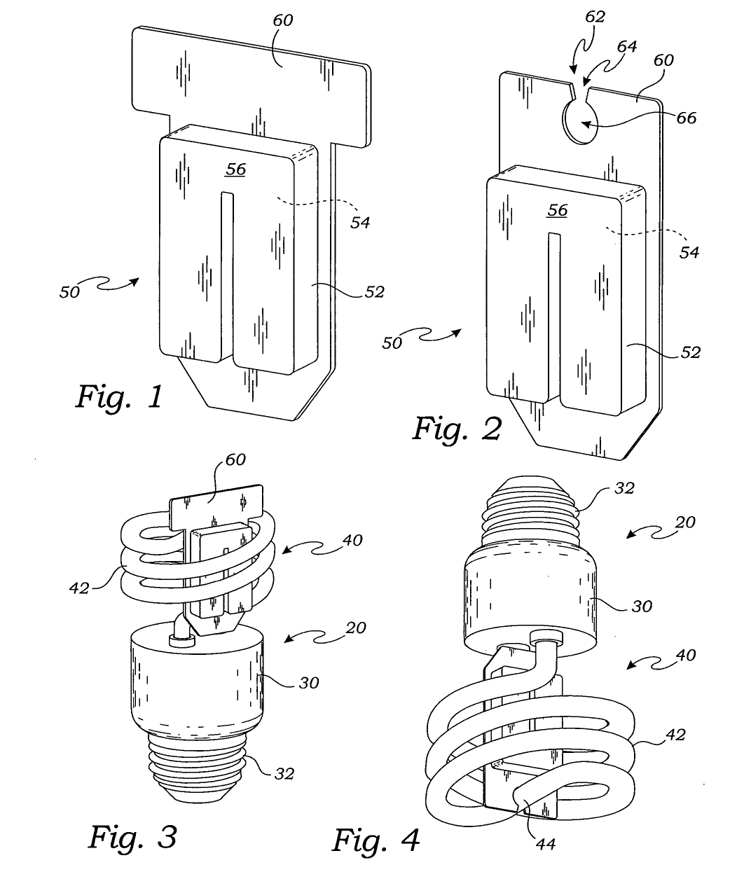

In one embodiment of the present invention, shown in FIG. 1, the fragrance producing packet 50 provides a tab 60 that extends laterally to each side of the pouch 52 so as to be adapted for diametrically resting on the coil portion 42 of the lamp 40 thereby positioning the pouch 52 within the coil portion 42 where it is exposed to the heat generated by the lamp 40. This is shown in FIG. 3 and is appropriate when the coil portion of the lamp is upright as shown.

In an alternate embodiment of the packet 50, shown in FIG. 2, the tab 60 provides a means for gripping 62 of the linear portion 44 of the lamp 40, thereby positioning the pouch 52 within the lamp coil portion 42, as shown in FIG. 4. Preferably, the gripping means 62 is a slot in the tab 60, the slot providing a slot entryway 64 having a width inferior, or more narrow than the linear portion 44 of the lamp 40. Preferably, the entryway 64 terminates within the tab 60 with a slot proper 66 having a width that is wider or superior to the linear portion 44. Such a tab 60 is able to be engaged with the linear portion 44 of the lamp so that the packet 50 is captured within the lamp 40 and will remain in place even when the lamp 40 is placed in the inverted attitude as shown in FIG. 4.

Of course, there is no reason why the tab 60 could not have both the laterally extended portions as well as the gripping means 62.

The enablements described in detail above are considered novel over the prior art of record and are considered critical to the operation of at least one aspect of one best mode embodiment of the instant invention and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or elements of the embodiments of the herein described invention and its related embodiments not described are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the invention and its various embodiments or that a single element may be substituted for two or more elements in a claim.

Changes from the claimed subject matter as viewed by a person with ordinary skill in the art, now known or later devised, are expressly contemplated as being equivalents within the scope of the invention and its various embodiments. Therefore, obvious substitutions now or later known to one with ordinary skill in the art are defined to be within the scope of the defined elements. The invention and its various embodiments are thus to be understood to include what is specifically illustrated and described above, what is conceptually equivalent, what can be obviously substituted, and also what essentially incorporates the essential idea of the invention.

While the invention has been described with reference to at least one preferred embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

1. A fragrance producing apparatus for engaging a lamp socket, the apparatus comprising in combination: an illuminating device having at a proximal end thereof, a base adapted for engaging the lamp socket, and a lamp extending from the base, the lamp providing a cylindrical coil portion; and a fragrance producing packet having a pouch holding a supply of a fragrance agent; the fragrance producing packet positioned within the coil portion and held there by a tab extending laterally with respect to the pouch so as to rest on the coil portion wherein the fragrance agent is heated by the lamp when the illuminating device is lit, the fragrance producing packet adapted for reacting to heat by slowly emitting the fragrance agent.

2. The apparatus of claim 1 wherein the pouch of the fragrance producing packet provides a thin wall permeable to the fragrance agent.

3. The apparatus of claim 1 wherein the tab diametrically rests on the coil portion thereby positioning the pouch within the coil portion.

4. (canceled)

5. The apparatus of claim 4 wherein the gripping means is a slot in the tab, the slot providing a slot entryway of width inferior to the linear portion of the lamp, the entryway terminating within the tab with a slot proper of width superior to the linear portion.

6. A fragrance producing apparatus comprising in combination: a cylindrical electric heating device formed as a spiral coil; and a fragrance producing element holding a supply of a fragrance agent; the fragrance producing element engaged by gripping the spiral coil within the electric heating device, wherein the fragrance agent is liberated by the heating device.

7. The apparatus of claim 6 wherein the fragrance producing element provides a tab, the tab adapted for diametrically resting on the cylindrical electric heating device.

8. The apparatus of claim 6 wherein the fragrance producing element provides a tab having formed therein, a means for gripping a linear portion of the electric heating device.

9. The apparatus of claim 8 wherein the gripping means is a slot in the tab, the slot providing a slot entryway of width inferior to the linear portion of the heating device, the entryway terminating within the tab with a slot proper of width superior to the linear portion.
